(19) United States
(12) Patent Application Publication
(10) Pub. No.: US 2011/0158954 A1
(43) Pub. Date: Jun. 30, 2011

(54) METHOD FOR PRODUCING GAMMA DELTA T CELL POPULATION

(76) Inventors: Mitsuko Ideno, Shiga (JP); Fumiyo Sakai, Shiga (JP); Tatsuji Enoki, Shiga (JP); Ikunoshin Kato, Shinga (JP)

(21) Appl. No.: 12/450,017

(22) PCT Filed: Mar. 4, 2008
(86) PCT No.: PCT/JP2008/053855

§ 371 Date: Sep. 8, 2009

(30) Foreign Application Priority Data
Mar. 9, 2007 (JP) 2007-061039

(51) Int. Cl. A61K 35/12; A61P 31/04; A61P 31/12; C12N 5/0783; C12N 15/86; C12N 15/867; C12N 15/861

(52) U.S. Cl. 424/93.7; 435/375; 435/456; 435/325

(57) ABSTRACT

A method for preparing a γδ T cell population, characterized in that the method includes the step of culturing a cell population containing γδ T cells, in the presence of (a) fibronectin, a fibronectin fragment or a mixture thereof and (b) an activating factor of γδ T cells. According to the present invention, a method for preparing a γδ T cell population is provided. The γδ T cell population obtained by the method is suitably used in, for example, immunotherapy. Therefore, the method of the present invention is expected to greatly contribute to a medical field.

### TECHNICAL FIELD

The present invention relates to a method for preparing a γδ T cell population, which is useful in the field of medicine.

### BACKGROUND ART

A living body is protected from foreign substances mainly by an immune response, and an immune system is constituted by various cells and the soluble factors produced thereby. Among the immune system, leukocytes, especially lymphocytes, play a key role. The lymphocytes are classified in two major types, B lymphocyte (which may be hereinafter referred to as B cell) and T lymphocyte (which may be hereinafter referred to as T cell), both of which specifically recognize an antigen and act on the antigen to protect the living body.

T cells are subclassified to αβ T cells and γδ T cells. αβ T cells have a αβ T cell receptor which recognizes an antigen peptide binding to a major histocompatibility complex (MHC) I and II molecule, and it is said that these cells occupy about from 90 to 98% of the T cells. On the other hand, γδ T cells have a γδ T cell receptor, and it is said that these cells occupy 3 to 5% of the T cells.

It is known that γδ T cells play an important role in a protection and an autoimmune against bacterial and viral infections, and that the cells proliferate when the living body is infected with an infective disease (for example, tuberculosis, salmonellosis, malaria, or the like). It is known that γδ T cells recognize an antigenic ligand of antigens by a direct interaction with them, without presentation of MHC molecules by antigen presenting cells. In other words, the γδ T cells exhibit a potent MHC non-restricted cytotoxic activity upon activation, which is effective in killing of various cell types, especially in killing pathogenic cells.

In a pathologic condition of cancer, immunotherapy has drawn an interest in recent years, as a fourth therapy following surgery, chemotherapy, and radiotherapy. Since immunotherapy utilizes immunocompetence inherently owned by a human, it is said that a physical burden on a patient caused by immunotherapy is light as compared to that caused by other therapies. As immunotherapy, a therapy including the step of transferring lymphokine-activated cells, NKT cells, γδ T cells, or the like obtained by expansion of peripheral blood lymphocytes or T cells induced ex vivo, or the like according to various methods; a dendritic cell-transfer therapy or a peptide vaccine therapy by which an induction of antigen-specific CTLs in vivo is expected; Th1 cell therapy; immune gene therapy further including the step of transducing a gene for which various effects can be expected to the above-mentioned cells ex vivo and transferring a transduced cell to the body; and the like, have been known.

As to the method for preparing γδ T cells ex vivo or in vitro, there has so far been some reports. For example, a method including the steps of subjecting lymphocytes to primary culture in the presence of interleukin-12 and CD2 ligand, and subjecting the lymphocytes to secondary culture in the presence of T cell mitogen and interleukin-2 has been disclosed (for example, Patent Publication 1). In addition, various compounds activating the γδ T cells have been studied. For example, phosphohalohydrins, phosphoepoxides, bisphosphonate compounds, isopentenyl pyrophosphate, or the like have been known (for example, Patent Publications 2 to 4, Non-Patent Publication 1).

Fibronectin is a gigantic glycoprotein having a molecular weight of 250 thousands, which exists in an animal blood, on the surface of a cultured cell, or in an extracellular matrix of a tissue, and has been known to have various functions. A domain structure thereof is divided into seven portions (FIG. 1 et seq), wherein three kinds of similar sequences are contained in an amino acid sequence thereof, repetitions of each of these sequences constituting the entire sequence. Three kinds of the similar sequences are referred to as type I, type II and type III. Among them, the type III is constituted by 71 to 96 amino acid residues, wherein an identity of these amino acid residues is from 17 to 40%. In fibronectin, there are fourteen type III sequences, among which the 8th, 9th and 10th sequences (each being hereinafter referred to as III-8, III-9 and III-10) are contained in a cell binding domain, and the 12th, 13th and 14th sequences (each being hereinafter referred to as III-12, III-13 and III-14) are contained in a heparin binding domain. In addition, a VLA (very late activation antigen)-5 binding region is contained in III-10, and its core sequence is RGDS. In addition, a region referred to as IIICS exists at a C-terminal side of the heparin binding domain. A region referred to as CS-1 consisting of 25 amino acids and having a binding activity to VLA-4 exists in IIICS (for example, Non-Patent Publications 2 to 4).

Among the immunotherapies, in a therapy including the step of transferring lymphokine-activated cells obtained by expanding CTLs or peripheral blood lymphocytes induced ex vivo, or the like using an action of IL-2 and anti-CD3 antibody, regarding problems such as how a cytotoxic activity is maintained when the antigen-specific CTLs induced ex vivo are expanded, or how lymphocytes can be effectively expanded ex vivo, the present inventors have already studied the effects obtained by using fibronectin or a fibronectin fragment (for example, Patent Publications 5 to 7).
Non-Patent Publication 1: Kunzmann V. and five others, Blood., 2000, 96(2), 384-392
Non-Patent Publication 2: FIBRONECTIN, ACADEMIC PRESS INC., 1-8, authored by Deane F. Momer, published in 1988
Non-Patent Publication 3: Kimizuka F. and eight others, J. Biochem., 1991, 110(2), 284-291
Non-Patent Publication 4: Hanenberg H. and five others, Human Gene Therapy, 1997, 8(18), 2193-2206
Patent Publication 1: WO 99/46365
Patent Publication 2: WO 00/12516
Patent Publication 3: WO 00/12519
Patent Publication 4: U.S. Pat. No. 5,639,653
Patent Publication 5: WO 03/016511
Patent Publication 6: WO 03/080817
Patent Publication 7: WO 2005/019450

### DISCLOSURE OF THE INVENTION

### Problems to be Solved by the Invention

An object of the present invention is to provide a method for preparing a γδ T cell population, which is effective in administration to a living body.

### Means to Solve the Problems

A first invention of the present invention relates to a method for preparing a γδ T cell population, characterized in that the method includes the step of culturing a cell population containing γδ T cells, in the presence of (a) fibronectin, a fibronectin fragment or a mixture thereof and (b) an activating factor of γδ T cells. In the first invention of the present invention, the step of culturing a cell population containing γδ T cells in the presence of fibronectin, a fibronectin fragment or a mixture thereof is exemplified by a step which is carried out in the presence of IL-2. In addition, a fibronectin fragment is exemplified by a polypeptide (m) containing at least any one of the amino acid sequences shown in SEQ ID NOs: 1 to 8 of Sequence Listing, or a polypeptide (n) containing at least one the amino acid sequence having substitution, deletion, insertion or addition of one or the plural number of amino acids in any one of the amino acid sequences, wherein the polypeptide (n) has a function equivalent to that of the polypeptide (m). In addition, a fibronectin fragment is exemplified by a polypeptide containing all of the amino acid sequences shown in SEQ ID NOs: 5 to 8 of Sequence Listing. In addition, a fibronectin fragment is exemplified by a polypeptide containing any one of the amino acid sequences shown in SEQ ID NOs: 9 to 22 of Sequence Listing. In addition, in the first invention of the present invention, the activating factor of γδ T cells is exemplified by a bisphosphonic acid compound and/or a pyrophosphate monoester compound, wherein the bisphosphonic acid compound is exemplified by at least one compound selected from the group consisting of pamidronate, alendronate, zoledronate, risedronate, neridronate, ibandronate, incadronate, olpadronate, solvadronate, minodronate, EB1053, etidronate, clodronate, tiludronate and medronate, and wherein the pyrophosphate monoester compound is exemplified by at least one compound selected from the group consisting of isopentenyl pyrophosphate, 2-methyl-3-butenyl-1-pyrophosphate and 4-hydroxy-3-methyl-2-butenyl-1-pyrophosphate. In addition, the first invention of the present invention exemplifies a method for preparing a γδ T cell population, further including the step of transducing a foreign gene into the cell population. The foreign gene can be transduced using retrovirus vector, adenovirus vector, adeno-associated virus vector, lentivirus vector or Simian virus vector.

A second invention of the present invention relates to a γδ T cell population obtained by the first method of the present invention.

A third invention of the present invention relates to a pharmaceutical agent containing as an effective ingredient the γδ T cell population obtained by the first method of the present invention.

A fourth invention of the present invention relates to a method for treating or preventing a disease, including the step of administering to a subject an effective amount of the γδ T cell population obtained by the first method of the present invention.

A fifth invention of the present invention relates to use of a γδ T cell population obtained by the first method of the present invention for the manufacture of a pharmaceutical agent.

A sixth invention of the present invention relates to a γδ T cell population obtained by the first method of the present invention for use in adoptive immunotherapy.

### Effects of the Invention

According to the present invention, there is provided a method for preparing a γδ T cell population having a high expansion fold. Since the γδ T cell population obtained by the preparation method has a high cytotoxic activity, it is highly useful in the treatment of a disease with cell therapy.

### BEST MODE FOR CARRYING OUT THE INVENTION

In the present invention, it has been found that a γδ T cell population having a very high expansion fold and an even higher cytotoxic activity is obtained by culturing a cell population containing γδ T cells in the presence of (a) fibronectin, a fibronectin fragment or a mixture thereof, and (b) an activating factor of γδ T cells. The present invention has been perfected thereby.

Incidentally, a γδ T cell population obtained by the preparation method of the present invention as used herein means a T cell population that contains γδ T cells in a high ratio. Also, a high ratio as used herein means that the ratio of the γδ T cells is high, as compared to a cell population that contains γδ T cells used in the preparation method of the present invention.

The present invention will be concretely explained hereinbelow.

(1) Fibronectin and Fibronectin Fragment Used in the Present Invention

The fibronectin described herein may be either those obtained from nature, or those artificially synthesized. The fibronectin can be prepared in a substantially pure form from a substance of natural origin, on the basis of the disclosure, for example, of Ruoslahti E., et al. [J. Biol. Chem., 256(14), 7277-7281 (1981)]. Here, the term “substantially pure fibronectin” described herein means that the fibronectin does not essentially contain other proteins existing together with fibronectin in nature.

Incidentally, it is known that there are a large number of splicing variants of fibronectin. As the fibronectin used in the present invention, any of variants can be used so long as the desired effects of the present invention are exhibited. For example, in the case of fibronectin derived from plasma, it is known that a region referred to as ED-B present in upstream of a cell binding domain and a region referred to as ED-A present between the cell binding domain and the heparin binding domain are deleted. Such fibronectin derived from plasma can also be used in the present invention. The above-mentioned fibronectin can be used in the present invention alone, or in a mixture of plural kinds.

In the present invention, a fibronectin fragment means an artificially prepared fragment (also referred to as a modified fibronectin fragment) which contains portions of an amino acid sequence (for example, 3 or more amino acids, preferably 10 or more amino acids, and more preferably 20 or more amino acids) of fibronectin. The fibronectin fragment is not particularly limited, so long as the fragment contains one or several parts of the amino acid sequence of the fibronectin, embracing a fragment of a part of naturally occurring fibronectin per se, or a fragment containing an amino acid sequence derived from those other than the above fragment and fibronectin. Incidentally, the useful information on the fibronectin fragments which can be used in the present invention and the preparation of the fragments can be obtained from Kimizuka F., et al. [J. Biochem., 110, 284-291 (1991)], Kornbrihtt A. R., et al. [EMBO J., 4(7), 1755-1759 (1985)], Sekiguchi K., et al. [Biochemistry, 25(17), 4936-4941 (1986)], and the like. In addition, the nucleic acid sequence encoding fibronectin or the amino acid sequence of fibronectin is disclosed in GenBank Accession No. NM_{—}002026 and NP_{—}002017.

In the present invention, the fibronectin fragment is exemplified by, for example, a polypeptide (m) containing at least one amino acid sequence containing any of the regions of III-8 (amino acid sequence shown in SEQ ID NO: 1 of Sequence Listing), III-9 (amino acid sequence shown in SEQ ID NO: 2 of Sequence Listing), III-10 (amino acid sequence shown in SEQ ID NO: 3 of Sequence Listing), III-11 (amino acid sequence shown in SEQ ID NO: 4 of Sequence Listing), III-12 (amino acid sequence shown in SEQ ID NO: 5 of Sequence Listing), III-13 (amino acid sequence shown in SEQ ID NO: 6 of Sequence Listing), III-14 (amino acid sequence shown in SEQ ID NO: 7 of Sequence Listing), and CS-1 (amino acid sequence shown in SEQ ID NO: 8 of Sequence Listing) (see FIG. 1), and a polypeptide (n) containing at least one amino acid sequence having substitution, deletion, insertion or addition of one or a plural number of amino acids in any one of the amino acid sequences described above, wherein the polypeptide (n) has a function equivalent to that of the above-mentioned polypeptide (m). The length of the fragment is, for example, in terms of the number of amino acid residues, preferably from 20 to 1000, and more preferably from 100 to 800. Here, the plural number herein is a concept that includes the several numbers, and is preferably from 2 to 12, more preferably from 2 to 10, and further more preferably from 2 to 8, which is hereinafter referred to the same.

In addition, as the fragment, a fragment having a cell adhesion activity and/or a heparin binding activity can be preferably used. The cell adhesion activity can be evaluated by assaying binding of the fragment (its cell binding domain) used in the present invention to a cell using a known method. For example, the method as mentioned above includes a method of Williams D. A., et al. [Nature, 352, 438-441 (1991)]. The method is a method of determining the binding of a cell to a fragment immobilized to a culture plate. In addition, the heparin binding activity can be evaluated by assaying binding of the fragment (its heparin binding domain) used in the present invention to heparin using a known method. For example, the binding of the fragment to heparin can be evaluated in the same manner by using heparin, for example, a labeled heparin in place of the cell in the above-mentioned method of Williams D. A., et al.

Further, the fibronectin fragment is exemplified by a polypeptide selected from the group consisting of C-274 (amino acid sequence shown in SEQ ID NO: 9 of Sequence Listing), H-271 (amino acid sequence shown in SEQ ID NO: 10 of Sequence Listing), H-296 (amino acid sequence shown in SEQ ID NO: 11 of Sequence Listing), CH-271 (amino acid sequence shown in SEQ ID NO: 12 of Sequence Listing), CH-296 (amino acid sequence shown in SEQ ID NO: 13 of Sequence Listing), C-CS1 (amino acid sequence shown in SEQ ID NO: 14 of Sequence Listing), and CH-296Na (amino acid sequence shown in SEQ ID NO: 15 of Sequence Listing).

Each of the above-mentioned fragments CH-271, CH-296, CH-296Na, C-274 and C-CS1 is a polypeptide having a cell binding domain with a binding activity to VLA-5. Also, C-CS1, H-296, CH-296 and CH-296Na are polypeptide having CS-1 with a binding activity to VLA-4. Further, H-271, H-296, CH-271, CH-296 and CH-296Na are polypeptide having a heparin binding domain. Here, CH-296Na is a polypeptide containing a region from the cell binding domain to CS-1 of fibronectin derived from plasma.

In the present invention, a fragment in which each of the above domains is modified can also be used. The heparin binding domain of the fibronectin is constituted by three type III sequences (III-12, III-13 and III-14). A fragment containing a heparin binding domain having deletion of one or two of the above type III sequences can also be used in the present invention. For example, the fragments may be exemplified by CHV-89 (amino acid sequence shown in SEQ ID NO: 16 of Sequence Listing), CHV-90 (amino acid sequence shown in SEQ ID NO: 17 of Sequence Listing) and CHV-92 (amino acid sequence shown in SEQ ID NO: 18 of Sequence Listing), fragments in which a cell binding site of the fibronectin (VLA-5 binding region: Pro1239 to Ser1515) and one of the type III sequences are bound, or CHV-179 (amino acid sequence shown in SEQ ID NO: 19 of Sequence Listing) and CHV-181 (amino acid sequence shown in SEQ ID NO: 20 of Sequence Listing), fragments in which the cell binding site of the fibronectin and two of the type III sequences are bound. CHV-89, CHV-90 and CHV-92 contain III-13, III-14 and III-12, respectively, and CHV-179 contains III-13 and III-14, and CHV-181 contains III-12 and III-13, respectively.

In addition, a fragment having addition of an additional amino acid to each of the above-mentioned fragments can be used in the present invention. For example, the fragment can be prepared by adding a desired amino acid to each of the above-mentioned fragments. For example, H-275-Cys (amino acid sequence shown in SEQ ID NO: 21 of Sequence Listing) is a fragment having a heparin binding domain of the fibronectin, and cysteine residue at a C-terminal.

In addition, as a fibronectin fragment, a polypeptide overlappingly containing amino acid sequences shown in SEQ ID NOs: 1 to 8 of Sequence Listing can be used. For example, H296-H296 (amino acid sequences shown in SEQ ID NO: 22 of Sequence Listing), which is a polypeptide overlappingly containing the above-mentioned heparin binding domain and CS-1 domain is preferably used.

Here, the fragment used in the present invention may be those containing a polypeptide comprising an amino acid sequence having substitution, deletion, insertion or addition of one or the plural number of amino acids in an amino acid sequence of a polypeptide constituting the fragment containing at least a part of an amino acid sequence of naturally occurring fibronectin exemplified above, wherein the polypeptide has a function equivalent to that of the fragment, so long as the desired effects of the present invention are obtained.

It is preferable that the substitution or the like of the amino acids is made to an extent that it can change physicochemical characteristics and the like of a polypeptide within the range that the inherent function of the polypeptide can be maintained. For example, it is preferable that the substitution or the like of the amino acids is conservative, within the range that the characteristics inherently owned by the polypeptide (for example, hydrophobicity, hydrophilicity, electric charge, pK and the like) are not substantially changed. For example, it is preferable that the substitution of the amino acids is substitutions within each of the groups of: 1. glycine, alanine; 2. valine, isoleucine, leucine; 3. aspartic acid, glutamic acid, asparagine, glutamine; 4. serine, threonine; 5. lysine, arginine; 6. phenylalanine, tyrosine, and that deletion, addition or insertion of amino acids is deletion, addition or insertion of the amino acids having characteristics similar to the characteristics of the surroundings of the subject site in the polypeptide within the range that the characteristics of the surroundings of the subject site are not substantially changed.

Here, when the fragment used in the present invention has been obtained by genetic engineering technique, in a case where, for example, a polypeptide is prepared using Escherichia coli or the like as a host, methionine at an N-terminal is sometimes deleted by the effect of methionine peptidase or the like, derived from Escherichia coli, and the polypeptide as mentioned above can be also used in the present invention. In other words, a polypeptide having deletion of methionine at an N-terminal of the polypeptides shown in SEQ ID NOs: 15 and 21 of Sequence Listing can be also preferably used in the present invention.

The substitution or the like of the amino acids may be those naturally occurring being caused by difference between species or individuals, or may be those artificially induced. Artificial induction may be carried out by a known method and is not particularly limited. The artificial induction may be carried out by preparing, for example, a given nucleic acid having substitution, deletion, addition or insertion of one or the plural number of nucleotides in the nucleic acid encoding the above-mentioned region and the given fragment derived from naturally occurring fibronectin, in accordance with a known method, and using the nucleic acid, whereby a polypeptide containing an amino acid sequence having substitution or the like in the amino acid sequence of the polypeptide constituting the fragments and the like, having a function equivalent to that of the above-mentioned region and the given fragment derived from naturally occurring fibronectin can be prepared.

In addition, the phrase “having a function equivalent” herein refers to that the expansion fold of a γδ T cell population or the cytotoxic activity of γδ T cells, obtained by using the fibronectin or the fibronectin fragment mentioned above is higher than that of a γδ T cell population obtained in the absence of the fibronectin or the fibronectin fragment mentioned above, which is a comparative control. The above-mentioned action can be appropriately confirmed in accordance with the method and the like described in Examples 1 to 7 set forth below. In addition, as the fragment comprising a polypeptide having the substitution or the like of the amino acids, a fragment having a cell adhesion activity and/or a heparin binding activity is preferred, and a fragment having CS-1 domain is also preferred. The cell adhesion activity and the heparin binding activity can be evaluated in accordance with the above-mentioned methods for determining those activities.

As the fragment comprising a polypeptide having the substitution or the like of the amino acids, for example, a fragment having one or more amino acids inserted as a linker between two different domains can also be used in the present invention.

In addition, as the fibronectin or the fibronectin fragment used in the present invention, so long as the desired effects of the present invention are obtained, there can be used a polypeptide having an identity of 50% or more, preferably a polypeptide having an identity of 70% or more, more preferably a polypeptide having an identity of 90% or more, and even more preferably a peptide having an identity of 95% or more, to an amino acid sequence of a polypeptide constituting the fibronectin or the fibronectin fragment, wherein the polypeptide has a function equivalent to the naturally occurring fibronectin or a fragment containing at least a part of an amino acid sequence thereof exemplified above. Incidentally, for example, DNASIS Pro Ver. 2.6 (manufactured by TAKARA BIO INC.) can be used for the calculation of the identity.

Incidentally, the fibronectin fragment most preferably used in the present invention includes a polypeptide containing in the amino acid sequence all of at least III-12 (amino acid sequence shown in SEQ ID NO: 5 of Sequence Listing), III-13 (amino acid sequence shown in SEQ ID NO: 6 of Sequence Listing), III-14 (amino acid sequence shown in SEQ ID NO: 7 of Sequence Listing), and CS-1 (amino acid sequence shown in SEQ ID NO: 8 of Sequence Listing), in other words, a polypeptide containing both of a heparin binding domain and a CS-1 domain, and more preferably, the fibronectin fragment includes the above-mentioned CH-296, H296-H296, or a polypeptide comprising an amino acid sequence having substitution, deletion, insertion or addition of one or the plural number of amino acids in an amino acid sequence of the polypeptide constituting the fragment, having a function equivalent to that of those polypeptides.

The fibronectin fragment described herein can be also prepared from a genetic recombinant on the basis of, for example, the description of the specification of U.S. Pat. No. 5,198,423. For example, each of the above-mentioned fragments of H-271 (SEQ ID NO: 10), H-296 (SEQ ID NO: 11), CH-271 (SEQ ID NO: 12) and CH-296 (SEQ ID NO: 13) and a method of obtaining these fragments are described in detail in the specification of this patent. In addition, CH-296Na (SEQ ID NO: 15) and the preparation method thereof are described in WO 2005/019450. In addition, the above-mentioned C-274 (SEQ ID NO: 9) fragment can be obtained in accordance with the method described in the specification of U.S. Pat. No. 5,102,988. Further, the C-CS1 (SEQ ID NO: 14) fragment can be obtained in accordance with the method described in the specification of Japanese Patent Gazette No. 3104178. Each of the above-mentioned fragments of CHV-89 (SEQ ID NO: 16), CHV-90 (SEQ ID NO: 17) and CHV-179 (SEQ ID NO: 19) can be obtained in accordance with the method described in the specification of Japanese Patent Gazette No. 2729712. In addition, the CHV-181 (SEQ ID NO: 20) fragment can be obtained in accordance with the method described in WO 97/18318. The CHV-92 (SEQ ID NO: 18) fragment can be obtained by genetic engineering technique using a plasmid constructed in a conventional manner on the basis of the plasmid described in the literatures by referring to the specification of Japanese Patent Gazette No. 2729712 and WO 97/18318.

These fragments or fragments which can be derived from these fragments in a conventional manner can be prepared by using microorganisms deposited to the International Patent Organism Depositary, National Institute of Advanced Industrial Science and Technology, Tsukuba Central 6, 1-1, Higashi 1-chome, Tsukuba-shi, Ibaraki-ken, Japan (Zip code 305-8566) under the following accession numbers, or also prepared by modifying a plasmid carried in each microorganism in accordance with a known method.

FERM BP-2264 (Escherichia coli carrying a plasmid encoding H-271, Date of Deposit: Jan. 30, 1989);
FERM BP-2800 (Escherichia coli carrying a plasmid encoding CH-296, Date of Deposit: May 12, 1989);
FERM BP-2799 (Escherichia coli carrying a plasmid encoding CH-271, Date of Deposit: May 12, 1989);
FERM BP-7420 (Escherichia coli carrying a plasmid encoding H-296, Date of Deposit: May 12, 1989);
FERM BP-1915 (Escherichia coli carrying a plasmid encoding C-274, Date of Deposit: Jun. 17, 1988);
FERM BP-5723 (Escherichia coli carrying a plasmid encoding C-CS1, Date of Deposit: Mar. 5, 1990);
FERM BP-10073 (plasmid encoding CH-296Na, Date of Deposit: Jul. 23, 2004);
FERM P-12182 (Escherichia coli carrying a plasmid encoding CHV-89, Date of Deposit: Apr. 8, 1991); and
FERM P-12183 (Escherichia coli carrying a plasmid encoding CHV-179, Date of Deposit: Apr. 8, 1991).
FERM P-20602 (Escherichia coli carrying a plasmid encoding H296-H296, Date of Deposit: Jul. 22, 2005).

Since the fibronectin is a gigantic glycoprotein, it is not necessarily easy industrially and in the manufacture of the pharmaceutical agent to prepare and use a naturally occurring protein. In addition, since the fibronectin is a multifunctional protein, there may be considered some disadvantages caused by a region different from the region exhibiting the effect by the method of the present invention depending on the conditions of its use. For these reasons, it is preferable that a fibronectin fragment is preferably used in the present invention, from the viewpoint of availability, easy handling, and safety. In addition, it is preferable to use the above-mentioned fibronectin fragment, from the viewpoint of realizing a high expansion fold. In addition, the molecular weight of the fibronectin fragment used in the present invention is not particularly limited, and is, for example, from 1 to 230 kD, preferably from 1 to 200 kD, more preferably from 5 to 190 kD, even more preferably from 5 to 180 kD, and still even more preferably from 10 to 180 kD. The molecular weight can be determined, for example, by SDS-polyacrylamide gel electrophoresis.

Here, in the amino acid sequence of the polypeptide constituting the fibronectin fragment of the present invention, the part of amino acid sequence other than the amino acid sequence of the polypeptide derived from the fibronectin is arbitrary and is not particularly limited, so long as the exhibition of the desired effects of the present invention is not inhibited.

(2) Method for Preparing γδ T Cell Population

The method for preparing a γδ T cell population of the present invention will be concretely explained hereinbelow. The present invention is a method for preparing a cell population that contains γδ T cells in a high ratio. The method of the present invention is characterized in that the method includes the step of culturing a cell population containing γδ T cells, in the presence of the (a) fibronectin, a fibronectin fragment or a mixture thereof (which may be hereinafter referred to as a component (a)), and (b) an activating factor of γδ T cells (hereinafter, which may be referred to as a (b) component) as mentioned above. The preparation method of the present invention has a high expansion fold of γδ T cells. Also, a γδ T cell population obtained by the method has a very useful characteristic of having a high cytotoxic activity. Incidentally, the term “mixture thereof” as used herein means a mixture of two or more kinds of materials selected from the group consisting of the fibronectin and the above-mentioned fibronectin fragments, which means a mixture of the fibronectin and one or more kinds of the above-mentioned fibronectin fragments or a mixture of two or more kinds of the above-mentioned fibronectin fragments.

The cell population containing γδ T cells used in the preparation method of the present invention is exemplified by PBMCs, hematopoietic stem cells, umbilical cord blood mononuclear cells, or the like. Incidentally, PBMCs as used herein mean peripheral blood derived-mononuclear cells, and, for example, PBMCs can be isolated and obtained from blood obtained by collecting blood, or a blood component obtained by apheresis by a method such as density gradient centrifugation. Here, upon the obtainment of the PBMCs, there is no particular limitation. Precursor cells and the like contained in a tissue fluid, a bone marrow aspirate, or the like can be also utilized. In addition, as the above-mentioned cell population containing γδ T cells, hematopoietic cells can be used in the present invention, so long as the hematopoietic cells contain γδ T cells. For example, blood such as peripheral blood and umbilical cord blood, blood from which components such as erythrocytes and plasmas are removed, a bone marrow aspirate, or the like can be used. In addition, as the cell population containing the γδ T cell population, any of the cell populations which are collected from a living body can be used, or those obtained via culture ex vivo, for example, the γδ T cell population obtained by the method of the present invention, can be used directly, or after being subjected to frozen storage. In addition, for example, γδ T cells obtained via various separation procedures from the PBMCs, hematopoietic stem cells, umbilical cord blood mononuclear cells, or the like obtained from a living body, or a T cell population of which γδ T cell ratio is enhanced by giving stimulation with an activating factor of γδ T cells described later can be used. Incidentally, in the present invention, the expansion of γδ T cells in a high ratio can be carried out by using a cell population containing γδ T cells as a part of a component cell. Therefore, as the cell population containing γδ T cells used in the present invention, PBMCs, hematopoietic stem cells, and umbilical cord blood mononuclear cells mentioned above are preferable.

In the present invention, as the activating factor of γδ T cells, a known compound having an action of activating or proliferating γδ T cells can be used. The activating factor is not particularly limited, and is exemplified by, for example, a bisphosphonic acid compound such as pamidronate, alendronate, zoledronate, risedronate, neridronate, ibandronate, incadronate, olpadronate, solvadronate, minodronate, EB1053, etidronate, clodronate, tiludronate or medronate; a pyrophosphate monoester compound such as isopentenyl pyrophosphate, 2-methyl-3-butenyl-1-pyrophosphate or 4-hydroxy-3-methyl-2-butenyl-1-pyrophosphate; a phosphohalohydrin such as 3-(bromomethyl)-3-butanol-1-yl-diphosphoric acid (BrHPP), 3-(iodomethyl)-3-butanol-1-yl-diphosphoric acid (IHPP), 3-(chloromethyl)-3-butanol-1-yl-diphosphoric acid (ClHPP), 3-(bromomethyl)-3-butanol-1-yl-triphosphoric acid (BrHPPP), 3-(iodomethyl)-3-butanol-1-yl-triphosphoric acid (IHPPP), α,γ-di-[3-(bromomethyl)-3-butanol-1-yl]-triphosphoric acid (diBrHTP), or α,γ-di[3-(iodomethyl)-3-butanol-1-yl]-triphosphoric acid (diIHTP); a phosphoepoxide such as 3,4-epoxy-3-methyl-1-butyl-diphosphoric acid (Epox-PP), 3,4-epoxy-3-methyl-1-butyl-triphosphoric acid (Epox-PPP), or α,γ-di-3,4-epoxy-3-methyl-1-butyl-triphosphoric acid (di-Epox-TP); an aminobiphosphonate compound such as 1-hydroxy-3-(methylpentylamino)propylidene-biphosphonic acid; an antibody having a binding activity against γδ type T cell receptor (γδ TCR) such as a pan-δ monoclonal antibody.

In the method for preparing a γδ T cell population of the present invention, it is preferable that a total number of cultured days for obtaining the γδ T cell population to be prepared is, for example, from 2 to 60 days, preferably from 4 to 40 days, and more preferably from 6 to 30 days. In addition, the culturing step of a cell population containing a γδ T cell population in the presence of the component (a) and the component (b) mentioned above, that is carried out in the method for preparing a γδ T cell population of the present invention, is preferably carried out in the presence of the component (a) and the component (b) mentioned above, particularly preferably at least in an early stage of the entire culture period. It is preferable that the culture in the presence of the effective ingredient in the present invention is carried out more preferably at least at the initiation of the culture. The culture in the presence of the effective ingredient in the present invention may be carried out during the entire period of the culture period, or during any part of the period. In other words, the present invention encompasses those embodiments which include the above-mentioned step in a part of the steps of preparing γδ T cells. It is preferable that the culture in the presence of the component (a) and the component (b) is carried out for at least 6 hours or more, more preferably for 12 hours or more, and even more preferably for 24 hours or more, from the initiation of the culture. In addition, the culture other than the culturing step in the presence of the component (a) and the component (b) mentioned above in the method for preparing the γδ T cells of the present invention can be carried out in the presence of either of the component (a) and the component (b) mentioned above, or in the absence of the component (a) and the component (b) mentioned above. For example, the cell population obtained by the culturing step for a period of from 2 to 7 days in the presence of the component (a) and the component (b) mentioned above can be cultured for a period of from additional 4 to 14 days in the presence of the component (a) and the component (b), or in the absence of the component (a) and the component (b) mentioned above.

In the present invention, the concentration of the component (a) during the culture is not particularly limited, and is, for example, preferably from 0.0001 to 500 μg/mL, and particularly preferably from 0.001 to 500 μg/mL. Here, the concentration of the component (a) during the culture means a concentration obtained by dissolving the component in a medium, or a concentration to be present in a medium in which the component is immobilized to an appropriate carrier.

In the present invention, the concentration of the component (b) during the culture can be properly set depending on the activating factor of γδ T cells to be used, and is not particularly limited. The concentration is exemplified by, for example, from 0.001 to 1000 μM, preferably from 0.005 to 100 μM, and particularly preferably from 0.01 to 50 μM.

The medium used in the method for preparing a γδ T cell population of the present invention is not particularly limited, and a known medium prepared by mixing components necessary for expanding γδ T cells can be used. For example, a commercially available medium can be appropriately selected to be used. These media may contain cytokines, appropriate proteins, and other components in addition to the inherent constituents. The cytokines are exemplified by, for example, IL-2, IL-7, IL-12, IFN-γ, IFN-α, IFN-β, IL-15 and the like, and preferably, a medium containing IL-2 is used. The concentration of IL-2 (cytokines in general) in the medium is not particularly limited, and is, for example, preferably from 0.01 to 1×10⁵ U/mL, and more preferably from 0.1 to 1×10⁴ U/mL. Also, the appropriate proteins are exemplified by CD3 ligand or CD28 ligand, for example, an anti-CD3 antibody or an anti-CD28 antibody. The concentration of the component in the medium is not particularly limited, so long as the desired effects can be obtained. However, in the present invention, as described in the Examples set forth below, the expansion can be realized with γδ T cells in a high ratio, even in the absence of an anti-CD3 antibody. In other word, in the present invention, the culture is preferably carried out in the absence of an anti-CD3 antibody. Also, as other components, various mitogens, and the like which contribute to the activation of the T cells are exemplified.

Furthermore, in the culture, serum and plasma can be also added to the medium. The amounts of serum and plasma added to the medium are not particularly limited, and are exemplified by an amount of from exceeding 0% (v/v) to 20% (v/v), and the amounts of the serum and plasma used can be changed depending on the stage of culture. For example, the serum or plasma can be used by stepwise decreasing the concentration thereof. Incidentally, origin of the serum or plasma may be either autologous (meaning that the origin is the same as that of the cell cultured) or nonautologous (meaning that the origin is different from that of the cell cultured). Preferably, autologous serum or plasma can be used, from the viewpoint of safety. Also, the culture can be carried out without adding the serum or plasma to the medium.

The preparation of a γδ T cell population of the present invention is usually carried out in a medium containing given components in the presence of (a) fibronectin, a fibronectin fragment or a mixture thereof, and (b) an activating factor of the γδ T cells mentioned above. The number of cells at the initiation of culture used in the present invention is not particularly limited, and is exemplified by, for example, preferably from 1 cell/mL to 1×10⁸ cells/mL, more preferably from 1 cell/mL to 5×10⁷ cells/mL, and even more preferably from 1 cell/mL to 2×10⁷ cells/mL. In addition, the culture conditions are not particularly limited, and usual conditions used for cell culture can be employed. For example, cells can be cultured under the conditions of from 20° to 40° C., preferably 37° C. in the presence of CO₂ or the like. In addition, the medium can be diluted by adding a fresh medium to the cell culture solution, the medium can be exchanged, or the cell culture equipment can be exchanged, at appropriate intervals.

The cell culture equipment used in the method for preparing a γδ T cell population of the present invention is not particularly limited, and, for example, a petri dish, a flask, a bag, a large culture device, a bioreactor and the like can be used. Here, as a bag, a CO₂ gas-permeable bag for cell culture can be used. In addition, in a case where γδ T cell populations are industrially mass-cultured, a large-scaled culture device can be used. Furthermore, the culture can be carried out in either an open system or a closed system. Preferably, the culture is carried out in a closed system, from the viewpoint of safety of the resulting T cell population.

Incidentally, fibronectin, a fibronectin fragment or a mixture thereof, and an activating factor of γδ T cells mentioned above, cytokines, appropriate proteins, and other components may be dissolved in the medium to be co-present, or the above components may be immobilized to an appropriate solid phase, for example, a cell culture equipment (including any of those of an open system and a closed system), such as a petri dish, a flask or a bag, or to a cell culture carrier such as beads, a membrane or a slide glass, and used. The materials for those solid phases are not particularly limited, so long as the materials can be used for cell culture. The above-mentioned carrier is used by immersing the carrier in a culture medium in the cell culture equipment during the cell culture. When the above-mentioned components are immobilized to the above-mentioned carrier, the amount of the components immobilized is not particularly limited, so long as the desired effects can be obtained.

A method for immobilizing the above components to the solid phase is not particularly limited. For example, the components can be immobilized by contacting these substances in an appropriate buffer. For example, regarding the immobilization of the fibronectin fragment to the solid phase, the immobilization can be also carried out in accordance with the methods described in WO 97/18318 and WO 00/09168.

Once various components mentioned above are immobilized to the solid phase, the γδ T cell population can be easily separated from the effective ingredient or the like only by separating the T cell population from the solid phase after the T cell population is obtained by the method of the present invention, so that the contamination of the T cell population with the effective ingredient or the like can be prevented.

In addition, using the γδ T cell population obtained by the preparation method of the present invention, a γδ T cell population further containing γδ T cells in high ratio, or only γδ T cells can be separated. The separation procedures are not particularly limited, and the separation can be carried out according to a known method, for example, by using a cell sorter, magnetic beads, column, or the like.

In addition, the γδ T cells prepared by the method of the present invention are cloned, whereby the γδ T cells can be maintained as stable γδ T cells. Also, a γδ T cell population can be newly obtained according to the method of present invention or a known method, using the γδ T cell population obtained according to the method of the present invention.

Diseases showing the effects by administering the γδ T cell population prepared by the method of the present invention, in other words, the diseases showing a sensitivity to the γδ T cell population prepared by the method of the present invention are not particularly limited, and are exemplified by, for example, cancer, leukemia, malignant tumor, hepatitis, and infectious diseases caused by a virus such as influenza or HIV, a bacterium, or a fungus, for example, tuberculosis, MRSA, VRE, and deep-seated mycosis. In addition, when a foreign gene for gene therapy is further transduced thereinto as described below, the effects are also exhibited for the intended various genetic diseases and the like. The γδ T cell population prepared by the method of the present invention can also be utilized for donor lymphocyte infusion and the like for the purpose of prevention from an infectious disease after bone marrow transplantation or X-ray irradiation, and remission of relapsed leukemia.

Further, the present invention provides a γδ T cell population obtained by the above preparation method of the present invention mentioned above. In addition, the present invention provides a pharmaceutical agent (therapeutic agent) containing the T cell population as an effective ingredient. The above-mentioned pharmaceutical agent containing the T cell population is suitably used in immunotherapy. For example, the pharmaceutical agent can be used as a pharmaceutical agent for adoptive immunotherapy or donor lymphocyte infusion. In the immunotherapy, the γδ T cell population suitable for the treatment of a patient is administered to the patient by, for example, an administration method such as an intravenous, intra-arterial, subcutaneous, or intraperitoneal method by an injection or infusions. The pharmaceutical agent is very useful for use in the above-mentioned diseases and donor lymphocyte infusion. The pharmaceutical agent can be prepared as infusions, an injection, or the like, by, for example, blending the T cell population prepared by the method of the present invention as an effective ingredient with a known organic or inorganic carrier, an excipient, a stabilizing agent and the like suitable for parenteral administration, according to a method known in the pharmaceutical field. Incidentally, the amount of the γδ T cell population of the present invention contained in the pharmaceutical agent, the dose of the pharmaceutical agent, and conditions for the pharmaceutical agent can be appropriately determined according to the known immunotherapy. For example, the amount of the γδ T cell population of the present invention contained in the pharmaceutical agent is not particularly limited, and is exemplified by, for example, preferably from 1×10³ to 1×10¹¹ cells/mL, more preferably from 1×10⁴ to 1×10¹⁰ cells/mL, and even more preferably from 1×10⁵ to 1×10⁹ cells/mL. Also, the dose of the pharmaceutical agent of the present invention is not particularly limited, and is exemplified by, for example, preferably from 1×10⁵ to 1×10¹² cells/day, more preferably from 1×10⁶ to 5×10¹¹ cells/day, and even more preferably from 1×10⁶ to 1×10¹¹ cells/day, for adult per day. Further, an immunotherapy by the pharmaceutical agent can be used in combination with a known chemotherapy by administration of a drug or radiotherapy, or therapy by surgery. In addition, an administration of the pharmaceutical agent is expected to exhibit a higher therapeutic effect of the γδ T cells administered to living body by a coadministration of the above-mentioned activating factor of γδ T cells. Incidentally, another embodiment of the present invention provides a pharmaceutical kit containing both of the γδ T cell population obtained by the preparation method of the present invention and the above-mentioned activating factor of γδ T cells.

The method for preparing a γδ T cell population of the present invention can further include the step of transducing a foreign gene into the T cells. In other words, one embodiment of the present invention provides a method for preparing a γδ T cell population, further including the step of transducing a foreign gene into the T cell population. Here, the term “foreign gene” means a gene which is artificially transduced into γδ T cells to be transduced with the gene, and also encompasses a gene derived from the same species as the one from which γδ T cells to be transduced with the gene is derived.

By carrying out the preparation method of the present invention, the ability for proliferation of the cultured γδ T cells is enhanced. By combining the method for preparing γδ T cells of the present invention with the step of transducing a gene, increase in the gene-transducing efficiency is expected.

A means for transducing a foreign gene is not particularly limited, and an appropriate means can be selected from known methods for transducing a gene to be used. The step of transducing a gene can be carried out at any given point during the preparation of a γδ T cell population. For example, it is preferable to carry out the step simultaneously with or during the course of the above-mentioned preparation of the T cell population, or after the step, from the viewpoint of working efficiency.

As the above-mentioned method for transducing a gene, any of methods using a viral vector, and methods without using the vector can be employed in the present invention. The details of those methods have been already published in numerous literatures.

The above-mentioned viral vector is not particularly limited, and a known viral vector ordinarily used in the method for transducing a gene, for example, retroviral vector, lentiviral vector, adenoviral vector, adeno-associated viral vector, Simian viral vector, vaccinia viral vector, Sendai viral vector, or the like is used. Particularly preferably, as the viral vector, retroviral vector, adenoviral vector, adeno-associated viral vector, lentiviral vector, or Simian viral vector is used. As the above-mentioned viral vector, those lacking replication ability so that the viral vector cannot self-replicate in an infected cell are preferable. In addition, upon the gene transduction, a substance which improves gene-transducing efficiency, such as RetroNectin (registered trademark, manufactured by TAKARA BIO INC.) can be also used.

The retroviral vector and lentiviral vector are used for the purpose of gene therapy or the like because they can stably incorporate a foreign gene inserted therein into chromosomal DNA in the cell into which the vectors are to be transduced. Since the vectors have high infection efficiency to the cells during division and proliferation, the vectors are preferable for carrying out the gene transduction in the preparation step in the present invention.

As the method for transducing a gene without using a viral vector, for example, a method using a carrier such as liposome or ligand-polylysine, calcium phosphate method, electroporation method, particle gun method or the like can be used without limiting the present invention thereto. In this case, a foreign gene is transduced, which is incorporated into a plasmid DNA, linear DNA, or RNA.

The foreign gene to be transduced into a γδ T cell population in the present invention is not particularly limited, and any gene which is desired to be transduced into the above-mentioned cells can be selected. As the gene as described above, besides a gene encoding a protein (for example, enzymes, cytokines, receptors, or the like), for example, a gene encoding an antisense nucleic acid, siRNA (small interfering RNA) or a ribozyme can be used. In addition, an appropriate marker gene which allows for selection of cells into which a gene is transduced may be simultaneously transduced.

The above-mentioned foreign gene can be, for example, inserted into a vector, a plasmid or the like, so as to be expressed under the control of an appropriate promoter to be used. In addition, in order to achieve an efficient transcription of a gene, there may exist other regulating elements which cooperate with a promoter or a transcription initiation site, for example, an enhancer sequence or a terminator sequence in the vector. In addition, for the purpose of inserting a foreign gene into a chromosome of T cells to be transduced with a gene by homologous recombination, for example, a foreign gene may be arranged between flanking sequences comprising nucleotide sequences each having identity to nucleotide sequences located on both sides of the desired target insertion site of the gene in the chromosome. The foreign gene to be transduced may be one that is a naturally occurring or an artificially generated, or may be one in which DNA molecules having different origins from each other are bound by a known means such as ligation. Moreover, the foreign gene may be one having a sequence in which a mutation is introduced into a naturally occurring sequence depending upon its purpose.

Genes to be transduced are exemplified by a gene encoding a TCR recognizing a surface antigen of target cells and a gene encoding a chimeric receptor having an antigen-recognizing site of an antibody to the surface antigen of target cells and containing an partial region of TCR complex (CD3, a partial region thereof, or the like). Here, as TCR, appropriate TCR can be selected depending diseases to be treated without limitation of αβ type and γδ type.

Also, for example, a gene encoding an enzyme associated with the resistance to a drug used for the treatment of a patient with cancer or the like can be transduced into γδ T cells, thereby giving the T cells a drug resistance. If the γδ T cells as described above are used, immunotherapy and drug therapy can be combined, and therefore, higher therapeutic effects can be obtained. The drug resistance gene is exemplified by, for example, a multidrug resistance gene.

On the other hand, conversely to the above-mentioned embodiment, a gene to give sensitivity to a particular drug can be transduced into a γδ T cell population, thereby giving the T cells sensitivity to the drug. In this case, the T cells after being transplanted into a living body can be removed by administering the drug. The gene for giving sensitivity to a drug is exemplified by, for example, a thymidine kinase gene.

The present invention also provides a method for treating or preventing a disease, including the step of administering to a subject an effective amount of the γδ T cell population obtained by the above-mentioned method. The term “subject” as used herein is not particularly limited, and preferably refers to a patient with the disease described above, to which the T cell population prepared by the method of the present invention is administered, in other word a disease which shows sensitivity to the T cell population. In addition, the method of treatment is exemplified by adoptive immunotherapy or donor lymphocyte infusion therapy. Here, the activating factor of γδ T cells as mentioned above can be administered to a patient together with the T cell population.

In addition, the term “effective amount” as used herein is an amount of the above-mentioned T cell population exhibiting a therapeutic or prophylactic effect, in a case where the γδ T cell population obtained by the method as described above is administered to the above-mentioned subject, as compared to a subject to which the T cell population is not administered. The specific effective amount is properly set depending upon its administration form, administration method, purpose of use, and age, weight, symptom or the like of a subject, and is not constant. The T cell population may be preferably administered in the same effective amount as the above-mentioned pharmaceutical agent. The administration method is also not limited. For example, the T cell population may be administered by infusion, injection, or the like, in the same manner as in the case of the above-mentioned pharmaceutical agent.

In addition, the present invention also provides use of the γδ T cell population obtained by the above-mentioned method in the manufacture of a pharmaceutical agent. The method for manufacturing the pharmaceutical agent is carried out in the same manner as that for the above-mentioned pharmaceutical agent. Also, diseases to which the pharmaceutical agent is administered are not particularly limited, and are the same as those of the above-mentioned pharmaceutical agent. The pharmaceutical agent is exemplified by a pharmaceutical agent for the adoptive immunotherapy or the donor lymphocyte infusion.

In addition, the present invention provides use of the above-mentioned γδ T cell population in use for the adoptive immunotherapy or the donor lymphocyte infusion. An amount of the above-mentioned γδ T cell population used in the use is not particularly limited, and includes for example the amounts exemplified by an amount of the above-mentioned γδ T cell population contained in the pharmaceutical agent.

### EXAMPLES

The present invention will be more specifically described hereinbelow by the Examples, without intending to limit the scope of the present invention thereto.

### Preparation Example 1

### Preparation of CH-296

CH-296, a polypeptide comprising a cell binding domain, a heparin binding domain and a CS-1 domain of fibronectin, was prepared using Escherichia coli HB101/pCH102 (FERM BP-2800) in accordance with the descriptions of U.S. Pat. No. 5,198,423.

### Preparation Example 2

### Generation of H296-H296

Of the procedures described in the present specification, the basic procedures such as the generation of plasmids and the digestion with restriction enzymes were performed in accordance with the method described in Molecular Cloning: A Laboratory Manual 3rd edition, 2001, edited by T. Maniatis et al., published by Cold Spring Harbor Laboratory Press.

(1) Construction of Expression Vectors

Construction of H-296 Expression Vector

A polypeptide consisting of amino acids 278-574 (base numbers 835-1725) from the N-terminal of the amino acid sequence of CH-296 shown in SEQ ID NO: 13 of Sequence Listing is referred to as H-296, and in order that a modified fibronectin fragment in which two of these H-296's connected to each other (H296-H296) is allowed to express, an expression vector was constructed in the following manner. Reference is made to FIG. 2 hereinbelow.

First, from the nucleotide sequence of CH-296 shown in SEQ ID NO: 13 of Sequence Listing (see WO 03/080817), synthetic primers H296-NcoF and H296-HindR having nucleotide sequences shown in SEQ ID NOs: 23 and 24 of Sequence Listing were synthesized with a DNA synthesizer, and purified according to a conventional method. The above-mentioned synthetic primer H296-NcoF is a synthetic DNA having a recognized sequence with a restriction enzyme NcoI in the base numbers 11-16, and further having a nucleotide sequence corresponding to amino acid numbers 278-283 of the amino acid sequence of CH-296 (SEQ ID NO: 13) in the base numbers 13-30. In addition, the synthetic primer H296-HindR is a synthetic DNA having a recognized sequence with a restriction enzyme HindIII in the base numbers 11-16, and further having a nucleotide sequence corresponding to amino acid numbers 570-574 of the amino acid sequence of CH-296 (SEQ ID NO: 13) in the base numbers 20-34.

PCR was carried out using the above-mentioned synthetic primers. The reaction conditions for PCR are given hereinafter. Concretely, about 0.1 μg of pCH102 as a template DNA, 5 μL of 10× Ex Taq Buffer (manufactured by TAKARA BIO, INC.), 5 μL of a dNTP mixture (manufactured by TAKARA BIO, INC.), 10 pmol of a synthetic primer H296-NcoF, 10 pmol of a synthetic primer H296-HindR, and 0.5 U TaKaRa Ex Taq (manufactured by TAKARA BIO, INC.) were added together, and a sterile water was added thereto to make up a total volume of 50 μL. The above reaction mixture was set in TaKaRa PCR Thermal Cycler SP (manufactured by TAKARA BIO, INC.), and 30 cycles of reaction, each cycle comprising 94° C. for 1 minute, 55° C. for 1 minute, and 72° C. for 3 minutes, were performed.

After the termination of the reaction, 5 μL of the reaction mixture was electrophoresed with 1.0% (w/v) agarose gel, and a desired DNA fragment having a size of about 0.9 kbp was confirmed. The remainder of the reaction mixture for PCR was electrophoresed, and a fragment thereof was collected and purified, and the purified mixture was subjected to ethanol precipitation. The DNA collected after the ethanol precipitation was suspended in 10 μL of a sterile water, and subjected to a double digestion with a restriction enzyme NcoI (manufactured by TAKARA BIO, INC.) and a restriction enzyme HindIII (manufactured by TAKARA BIO, INC.), and the digest was electrophoresed with 1.0% (w/v) agarose gel, thereby extracting and purifying the NcoI and HindIII digest, to give an NcoI-HindIII-digested DNA fragment.

Next, in accordance with the methods described in Examples 1 to 6 of WO 99/27117, a pCold04NC2 vector (hereinafter, this pCold04NC2 vector is simply referred to as “pCold14 vector”) was prepared.

Next, the above-mentioned pCold14 vector was cleaved with the same restriction enzymes as those used in the preparation of the above-mentioned NcoI-HindIII-digested DNA fragment, and the terminals of the products were subjected to a dephosphorization treatment to prepare a product, and the product was mixed with the above-mentioned NcoI-HindIII-digestedDNA fragment, and ligated using a DNA ligation kit (manufactured by TAKARA BIO, INC.). Thereafter, Escherichia coli JM109 was transformed with 20 μL of the ligation reaction mixture, and the resulting transformant was grown on an LB medium (containing 50 μg/mL ampicillin) containing agar in a concentration of 1.5% (w/v).

The plasmid inserted with a desired DNA fragment was confirmed by sequencing, and this recombinant plasmid was referred to as pCold14-H296. This pCold14-H296 is a plasmid containing a nucleotide sequence encoding the amino acid sequence of the amino acid numbers 278-574 of CH-296.

(ii) Construction of H296-H296 Expression Vector

Next, from the nucleotide sequences published in WO 03/080817, a synthetic primer H296-NcoR having the nucleotide sequence as shown in SEQ ID NO: 25 of Sequence Listing was synthesized with a DNA synthesizer, and purified according to a conventional method. The above-mentioned synthetic primer H296-NcoR is a synthetic DNA having a recognized sequence with a restriction enzyme NcoI in the base numbers 10-15, and further having a nucleotide sequence corresponding to amino acid numbers 574-569 of the amino acid sequence of CH-296 (SEQ ID NO: 13) in the base numbers 17-34. PCR was performed using the above-mentioned synthetic primer and a primer (NC2-5′UTR) that anneal a 5′UTR moiety of NC2 vector as shown in SEQ ID NO: 26 of Sequence Listing. The reaction conditions for PCR are given hereinafter.

Concretely, about 0.1 μg of pCold14-H296 as a template DNA, 10 μL of 10× Pyrobest Buffer (manufactured by TAKARA BIO, INC.), 8 ΞL of the dNTP mixture (manufactured by TAKARA BIO, INC.), 20 pmol of NC2-5′UTR, 20 pmol of a synthetic primer H296-NcoR, and 5 U Pyrobest DNA Polymerase (manufactured by TAKARA BIO, INC.) were added together, and a sterile water was added thereto to make up a total volume of 100 μL. The above reaction mixture was set in the TaKaRa PCR Thermal Cycler SP (manufactured by TAKARA BID, INC.), and 30 cycles of reaction, each cycle comprising 96° C. for 1 minute and 68° C. for 4 minutes, were performed.

After the termination of the reaction, 5 μL of the reaction mixture was electrophoresed with 1.0% (w/v) agarose gel, and a desired DNA fragment having a size of about 0.9 kbp was confirmed. The remainder of the reaction mixture for PCR was collected and purified with a Bio-Rad column, and the purified mixture was subjected to ethanol precipitation. The DNA collected after the ethanol precipitation was suspended in 39 μL of a sterile water, to make up a total volume of the reaction mixture of 50 μL, the reaction mixture was digested with the restriction enzyme NcoI (manufactured by TAKARA BIO, INC.), and the digest was electrophoresed with 1.0% (w/v) agarose gel, thereby extracting and purifying an NcoI-NcoI digest, to give an NcoI-NcoI-digested DNA fragment.

Next, the pCold14-H296 prepared in (i) was digested with the restriction enzyme NcoI, and the terminals of the digest were subjected to a dephosphorization treatment to prepare a product, and the product was mixed with the above-mentioned NcoI-NcoI-digested DNA fragment, and ligated with the DNA ligation kit (manufactured by TAKARA BIO, INC.). Thereafter, Escherichia coli JM109 was transformed with 20 μL of the ligation reaction mixture, and the resulting transformant was grown on an LB medium (containing 50 μg/mL ampicillin) containing agar in a concentration of 1.5% (w/v).

The plasmid inserted with a desired DNA fragment was confirmed by sequencing, and this recombinant plasmid was referred to as pCold14-H296-H296. The plasmid was named and identified as pCold14-H296-H296, and has been deposited as FERM P-20602 to the International Patent Organism Depositary, National Institute of Advanced Industrial Science and Technology, Tsukuba Central 6, 1-1, Higashi 1-chome, Tsukuba-shi, Ibaraki-ken, Japan (Zip code 305-8566) since Jul. 22, 2005. This pCold14-H296-H296 is a plasmid containing two nucleotide sequences encoding the amino acid sequence of the amino acid numbers 278-574 of CH-296 connected with an amino acid “A” inserted therebetween. The amino acid sequence of the protein is shown in SEQ ID NO: 22 of Sequence Listing.

(2) Expression and Purification

Escherichia coli BL21 was transformed with pCold14-H296-H296 prepared in the above-mentioned (1), and the resulting transformant was grown on LB medium (containing 50 μg/mL ampicillin) containing agar having a concentration of 1.5% (w/v). The grown colonies were inoculated on 30 mL of LB liquid medium (containing 50 μg/mL ampicillin), and cultured overnight at 37° C. An entire amount of the cultured medium was inoculated on 3 L of the same LB medium, and cultured at 37° C. up to a logarithmic growth phase. Here, this culture was carried out using a 5 L minijar fermenter (manufactured by Biott) under the conditions of 120 rpm and Air=1.0 L/min. After the above-mentioned culture, the culture medium was cooled to 15° C., IPTG (manufactured by TAKARA BIO, INC.) was then added so as to have a final concentration of 1.0 mM, and the culture was carried out in this state at 15° C. for 24 hours to induce expression. Thereafter, the cells were harvested by centrifugation, and resuspended in about 40 mL of a cell disruption solution [50 mM Tris-HCl (pH 7.5), 1 mM EDTA, 1 mM DTT, 1 mM PMSF, 50 mM NaCl]. The cells were disrupted by ultrasonic disruption, and the disrupted solution was centrifuged (11,000 rpm, 20 minutes), thereby separating the disrupted solution into an extract of the supernatant and precipitates. The extract of the supernatant obtained was dialyzed against 2 L of buffer A[50 mM Tris-HCl (pH 7.5), 50 mM NaCl], and subjected to further purification by ion-exchange chromatography using about 40 mL of the dialyzed extract in the manner described below.

Concretely, a column (φ4 cm×20 cm) of SP-Sepharose (manufactured by Amersham Pharmacia) having a resin volume corresponding to 100 mL, saturated with the buffer A[50 mM Tris-HCl (pH 7.5), 50 mM NaCl] was furnished, and the dialyzed sample was applied thereto. Thereafter, the elution of the target protein was carried out with a from 50 mM to 1 M sodium chloride concentration gradient in 250 mL of the buffer A and 250 mL of buffer B [50 mM Tris-HCl (pH 7.5), 1 M NaCl]. The fractionation was carried out in a 5 mL portion, and the fraction was analyzed by 10% (w/v) SDS-PAGE using acrylamide gel (hereinafter referred to as “SDS-PAGE”), to collect about 100 mL of a fraction richly containing a target protein having a molecular weight of about 64.6 kDa, and the fraction was dialyzed against 2 L of the buffer A.

Next, a column (φ 3 cm×16 cm) of Q-Sepharose (manufactured by Amersham Pharmacia) having a resin volume corresponding to 50 mL, saturated with the buffer A was furnished, and the dialyzed sample was applied thereto. Thereafter, the elution of the target protein was carried out with a 50 mM to 1 M sodium chloride concentration gradient in 250 mL of the buffer A and 250 mL of the buffer B. The fraction richly containing only a target protein was analyzed by 10% SDS-PAGE. As a result, the fraction was contained in non-adsorbent fraction, and about 100 mL of this fraction was collected, and dialyzed against 2 L of buffer D [50 mM sodium carbonate buffer (pH 9.5)].

Subsequently, the dialyzed fraction was concentrated about 20 times to a volume of 5 mL with Centricone-10 (manufactured by Millipore Corporation), and the concentrate was further confirmed by 10% SDS-PAGE. Consequently, a target protein having a molecular weight of about 64.6 kDa was detected in a nearly single band, which was referred to as H296-H296. Thereafter, a protein concentration was determined using a MicroBCA kit (manufactured by Pierce). As a result, the protein concentration was found to be 2.16 mg/mL (about 33.4 μM, calculated from its molecular weight). In addition, the N-terminal analysis was carried out; as a result, methionine was digested, so that the N-terminal was Ala.

### Example 1

### Expansion of γδ T Cell Population Using Pamidronate and Isopentenyl Pyrophosphate (IPP)

(1) Isolation and Storage of PBMCs

Apheresis was performed, or 50 mL blood was collected from human healthy donor obtained with informed consent. The collected blood was then diluted 2-folds with phosphate buffered saline (manufactured by NEXELL or SIGMA, hereinafter referred to as “PBS”), overlaid on Ficoll-paque (manufactured by GE Healthcare Bio-Science), and centrifuged at 600×g for 20 minutes. Peripheral blood mononuclear cells (hereinafter referred to as “PBMCs”) in an intermediate layer were collected with a pipette, and washed. The collected PBMCs were suspended in a storage solution composed of 90% (v/v) FBS (manufactured by MP Biomedicals, LLC)/10% (v/v) DMSO (manufactured by SIGMA), or a storage solution composed of equivolumes of CP-1 (manufactured by KYOKUTO PHARMACEUTICAL INDUSTRIAL CO., LTD.) containing 8% (w/v) human serum albumin (name of formulation: Buminate, manufactured by Baxter Limited, hereinafter referred as to “HSA”) and an RPMI 1640 medium (manufactured by SIGMA), and stored in liquid nitrogen. During expansion of γδ T cells, these stored PBMCs were rapidly melted in water bath at 37° C., and washed with RPMI 1640 medium containing 10 μg/mL DNase (manufactured by Calbiochem), 10% (v/v) human AB serum (manufactured by Cambrex), 2 mM L-glutamine (manufactured by Cambrex), and 100 μg/mL streptomycin sulfate (manufactured by MEIJI SEIKA KAISHA, LTD.) (hereinafter referred to as “10HRPMI+L-Gln”) or Iscove's Modified Dulbecco's Medium (IMDM) (manufactured by Invitrogen) containing 10 μg/mL DNase and 10% (v/v) human AB serum (hereinafter referred to as “10HIMDM”). Thereafter, the number of live cells was calculated by trypan blue staining method. The cells were subjected to each experiment.

(2) Immobilization of Fibronectin Fragments (CH-296 and H296-H296)

A fibronectin fragment (hereinafter referred to as a “FN fragment”) (CH-296 and H296-H296) was immobilized on a culture equipment used in the following experiment. Concretely, a buffer having a pH of 5.0 composed of 2.20% (w/v) sodium citrate dihydrate, 0.80% (w/v) citric acid monohydrate and 2.20% (w/v) glucose (all manufactured by Nakalai Tesque Inc.) (hereinafter referred to as “ACD-A solution (pH 5.0)”), containing CH-296 (final concentration: 25 μg/mL) or H296-H296 (final concentration: 3 μg/mL), was added to a 24-well cell culture plate (manufactured by Becton Dickinson or Corning), in a volume of 240 μL each.

These culture equipments was incubated for 5 hours or more at room temperature. Immediately before use, the ACD-A solution (pH 5.0) containing CH-296 or H296-H296 was removed by aspiration from these culture equipments, and thereafter each well was washed with twice PBS, and then once with RPMI 1640 medium, and the culture equipments were subjected to each experiment. As a control, a plate to which nothing was immobilized was used.

(3) Expansion of γδ T Cell Population

PBMCs prepared in Example 1-(1) were suspended in 10HRPMI+L-Gln or 10HIMDM so as to have a concentration of 1×10⁶ cells/mL, to prepare a cell suspension. Thereafter, the above-mentioned cell suspension was added to a plate to which nothing was immobilized or a plate immobilized with CH-296 or H296-H296, prepared in Example 1-(2), in a volume of 1 mL/well each. IL-2 (name of formulation: PROLEUKIN, manufactured by Chiron) was added to the plate so as to have a final concentration of 20 U/mL, and thereafter disodium pamidronate (pamidronate, name of formulation: Aredia injection: manufactured by NOVARTIS) (final concentration: 5 μM) or an ammonium isopentenyl pyrophosphate solution (IPP) (manufactured by SIGMA) (final concentration: 5 μM) was added thereto. These plates were subjected to culture at 37° C. in 5% CO₂ (zeroth day of culture).

On the fourth day from the initiation of culture, IL-2 was added to each well so as to have a final concentration of 20 U/mL. Upon the addition, the entire volume of the cell suspension was transferred to a fresh plate to which nothing was immobilized for stimulation conditions of CH-296 or H296-H296.

On the seventh day and the eleventh day from the initiation of culture, the cell suspension was diluted with a medium for culture so as to have a concentration of 0.5×10⁶ cells/mL, and the dilution was transferred to a 6-well or a 12-well cell culture plate (manufactured by Becton Dickinson, or Corning) to which nothing was immobilized. Thereafter, IL-2 was added to each well, so as to have a final concentration of 20 U/mL. The culture was continued up to the fourteenth day from the initiation of culture.

(4) Analysis of Ratio of T Cells Expressing γδ TCR

The ratio of γδ T cells was analyzed according to flow cytometry (Cytomics FC500, manufactured by Beckmann-Coulter) for PBMCs prepared in Example 1-(1) and the cells on the fourteenth day from the initiation of culture prepared in Example 1-(3). Concretely, the PBMCs or the cells on the fourteenth day from the initiation of culture were washed with PBS, and thereafter the cells were suspended in PBS containing 0.1% (w/v) bovine serum albumin (manufactured by SIGMA, hereinafter referred to as “BSA”) (hereinafter referred to as “0.1% (w/v) BSA/PBS”). FITC-labeled mouse anti-human γδ TCR (manufactured by Becton Dickinson) and PC5-labeled mouse anti-human CD3 antibody (manufactured by Beckmann-Coulter) were added to the cell suspensions. In the same manner, to a part of each of the cell population, as a negative control, FITC-labeled mouse IgG1/RD1-labeled mouse IgG1/PC5-labeled mouse IgG1 (manufactured by Beckmann-Coulter) was added. After the addition of each of the antibodies, the reaction mixture was incubated for 30 minutes on ice, and after the incubation, the cells were washed with 0.1% (w/v) BSA/PBS, and resuspended in PBS. These cells were subjected to flow cytometry, and the ratio of γδ T cells was calculated, supposing that a γδ TCR- and CD3-positive cells group is γδ T cells. As a result, the ratio of γδ T cells was 8.9% for PBMCs, and the ratio was from 30.7 to 52.0% for the cells on the fourteenth day from the initiation of culture.

(5) Expansion Fold of γδ T Cells

The number of live cells was counted by means of trypan blue staining method for the cells on the fourteenth day from the initiation of culture prepared in Example 1-(3). Using the results of the ratio of γδ T cells determined in Example 1-(4), the expansion fold was calculated according to the following formula in comparison of the counted number with the number of γδ T cells from the initiation of culture.

Expansion Fold of γδ T Cells=(Number of Live Cells on Fourteenth Day from Initiation of Culture×Ratio of γδ T Cells on Fourteenth Day from Initiation of Culture)/(Number of Live Cells at Initiation of Culture×Ratio of γδ T Cells at Initiation of Culture)

The results are shown in Table 1.

TABLE 1

| | | | Expansion Fold of |
|---|---|---|---|
| Medium for | | Immobilized FN | γδ T Cells (Folds) |
| Culture | Stimulant | Fragment | Fourteenth Day of Culture |
| 10HRPMI + | Pamid- | Control (Without | ×1.34 |
| L-Gln | ronate | Immobilization of | |
| | | FN Fragment) | |
| | | CH-296 | ×3.85 |
| | | H296-H296 | ×5.76 |
| | IPP | Control (Without | ×3.92 |
| | | Immobilization of | |
| | | FN Fragment) | |
| | | H296-H296 | ×16.27 |
| 10HIMDM | Pami- | Control (Without | ×2.03 |
| | dronate | Immobilization of | |
| | | FN Fragment) | |
| | | H296-H296 | ×3.18 |
| | IPP | Control (Without | ×0.67 |
| | | Immobilization of | |
| | | FN Fragment) | |
| | | H296-H296 | ×4.39 |

As shown in Table 1, the group in which the culture equipment immobilized with CH-296 or H296-H296 at an early stage of expansion of γδ T cells was used gave the results of higher expansion folds of γδ T cells, as compared to those of the control groups. The effects were obtained independently from the stimulant or the medium for culture. From these results, it was clarified that the γδ T cells obtained after the culture were increased by allowing the cells to co-exist with CH-296 or H296-H296 at an early stage of expansion.

### Example 2

### Expansion Fold of γδ T Cell Population Cultured by Using Pamidronate

(1) Expansion of γδ T Cell Population

PBMCs prepared in Example 1-(1) were suspended in IMDM containing 0.25% (w/v) HSA, so as to have a concentration of 2×10⁶ cells/mL, to prepare a cell suspension. Thereafter, the above-mentioned cell suspension was added to a plate to which nothing was immobilized or a plate immobilized with H296-H296, prepared in Example 1-(2), in a volume of 1 mL/well each. Pamidronate was added to each well so as to have a final concentration of 5 μM. These plates were incubated at 37° C. in 5% CO₂ (zeroth day of culture). On the second day from the initiation of culture, IL-2 was added to each well so as to have a final concentration of 100 U/mL. On the third day from the initiation of culture, a half the volume of culture supernatant was removed from each well, IMDM containing 20% (v/v) human AB serum was then added thereto in a volume of 500 μL/well each (final concentration of human AB serum: 10% (v/v)), and further IL-2 was added thereto so as to have a final concentration of 100 U/mL. On the fifth day from the initiation of culture, each group was diluted with 10HIMDM so as to have a concentration of from 0.6 to 0.9×10⁶ cells/mL, and thereafter IL-2 was added thereto so as to have a final concentration of 100 U/mL. On the eighth day and the eleventh day from the initiation of culture, each group was diluted with 10HIMDM so as to have a concentration of 1.0×10⁶ cells/mL, the cell suspension was then transferred to a 6-well or a 12-well cell culture plate to which nothing was immobilized, and IL-2 was added to each well so as to have a final concentration of 100 U/mL. The culture was continued up to the fourteenth day from the initiation of culture.

(2) Analysis of Ratio of γδ T Cells

The ratio of γδ T cells was analyzed according to flow cytometry for PBMCs prepared in Example 1-(1) and the cells on the fourteenth day from the initiation of culture prepared in Example 2-(1), in the same manner as in Example 1-(4). As a result, the ratio of γδ T cells was 8.9% for PBMCs, and the ratio was from 96.0 to 96.7% for the cells on the fourteenth day from the initiation of the culture.

(3) Expansion Fold of γδ T Cells

The number of live cells was counted according to trypan blue staining method for the cells on the fourteenth day from the initiation of culture prepared in Example 2-(1). The expansion fold of γδ T cells was calculated in comparison of the counted number with the number of γδ T cells at the initiation of culture, in the same manner as in Example 1-(5).

The results are shown in Table 2.

TABLE 2

| | | Expansion Fold of |
|---|---|---|
| | Immobilized | γδ T Cells (Folds) |
| Stimulant | FN fragment | Fourteenth Day of Culture |
| Pamidronate | Control (Without | ×74.1 |
| | Immobilization of | |
| | FN Fragment) | |
| | H296-H296 | ×101.8 |

As shown in the Table 2, the group in which the culture equipment immobilized with H296-H296 at an early stage of expansion of γδ T cells was used gave the result of a high expansion fold of γδ T cells, as compared to that of the control group.

### Example 3

### Determination of Cytotoxic Activity of γδ T Cell Population Cultured Using Pamidronate

(1) Expansion of γδ T Cell Population

The expansion was carried out in the same manner as Example 2, except that a plate immobilized with CH-296 prepared in Example 1-(2) was used.

(2) Analysis of Ratio of γδ T Cells

The ratio of the γδ T cells was analyzed according to flow cytometry for PBMCs prepared in Example 1-(1) and the cells on the fourteenth day from the initiation of culture prepared in Example 3-(1), in the same manner as in Example 1-(4). As a result, the ratio of γδ T cells was 8.9% for the PBMCs, and the ratio was from 96.0 to 96.9% for the cells on the fourteenth day from the initiation of culture.

(3) Expansion Fold of γδ T Cells

The number of live cells was counted according to trypan blue staining method for the cells on the fourteenth day from the initiation of culture prepared in Example 3-(1). The expansion fold of γδ T cells was calculated in comparison of the counted number with the number of γδ T cells at the initiation of culture, in the same manner as in Example 1-(5). As a result, it was confirmed that the group in which the culture equipment immobilized with CH-296 was used gave a high expansion fold of γδ T cells during the culture, as compared to that of the control group.

(4) Determination of Cytotoxic Activity

The cytotoxic activity was determined for the cells on the fourteenth day from the initiation of culture prepared in Example 3-(1). The cytotoxic activity was evaluated by a method for assaying cytotoxic activity using Calcein-AM [Lichtenfels R. et al., J. Immunol. Methods 172(2), 227-239 (1994)]. Concretely, K562 cells (Human Science Research Resource Bank JCRB0019), or Daudi cells (Human Science Research Resource Bank JCRB9071) were suspended in RPMI 1640 medium containing 5% (v/v) FBS so as to have a concentration of from 1 to 2×10⁶ cells/mL, and Calcein-AM (manufactured by DOJINDO LABORATORIES) was then added thereto so as to have a final concentration of 25 μM, and the cells were cultured at 37° C. for 1 hour. The cells that were washed with a medium without containing Calcein-AM were then used as Calcein-labeled target cells.

The cells on the fourteenth day from the initiation of culture prepared in Example 3-(1) were diluted with an RPMI 1640 medium containing 5% (v/v) human AB serum, 0.1 mM NEAA mixture, 1 mM sodium pyruvate (all manufactured by Cambrex), 2 mM L-glutamine, 10 μg/mL streptomycin sulfate (hereinafter referred to as “5HRPMI”) as effector cells, so as to have a concentration of 3×10⁶ cells/mL. Thereafter, the dilution was previously dispensed to each well of a 96-well cell culture plate (manufactured by Becton Dickinson or Corning) in a volume of 100 μL/well each, and the prepared Calcein-labeled target cells were added thereto in a volume of 100 μL/well each so that the Calcein-labeled target cells had a concentration of 1×10⁵/mL. Upon the addition, the ratio of the effector cells (E) to the Calcein-labeled target cells (T) is expressed as an E/T ratio, and the measurements were made at an E/T ratio of 30. The plate containing the above cell suspension was centrifuged at 400×g for 1 minute, and thereafter the cells were incubated in a 5% CO₂ incubator at 37° C. for 4 hours. Thereafter, 100 μL of the culture supernatant was collected from each well, and the amount of Calcein released into the culture supernatant was determined with a fluorescence plate reader (Mithras LB 940, manufactured by BERTHOLD TECHNOLOGIES) (excited at 485 nm/measured at 535 nm). The “cytotoxic activity (%)” was calculated in accordance with the following formula.

Cytotoxic Activity (%)={(Measured Value in Each Well−Minimum Released Amount)/(Maximum Released Amount−Minimum Released Amount)}×100

In the above formula, the minimum released amount is an amount of Calcein released in the well containing only the Calcein-labeled target cells, showing the amount of Calcein naturally released from the Calcein-labeled target cells. In addition, the maximum released amount refers to an amount of Calcein released when the cells are completely disrupted by adding a 0.1% (v/v) detergent Triton X-100 (manufactured by Nakalai Tesque Inc.) to the Calcein-labeled target cells. The measurement results of cytotoxic activity are shown in Table 3.

TABLE 3

Cytotoxic Activity (%)

| | | Control (Without | |
|---|---|---|---|
| | | Immobilization of | |
| Target Cells | E/T | FN Fragment) | CH-296 |
| K526 | 30 | 17.37 | 25.45 |
| Daudi | 30 | 25.34 | 33.23 |

As shown in Table 3, the group in which the culture equipment immobilized with CH-296 at an early stage of expansion of γδ T cells gave a high cytotoxic activity, as compared to the control group. In other words, it was clarified that cultured cells in which the culture equipment immobilized with CH-296 at an early stage of the expansion of the γδ T cells was used were γδ T cells having a higher cytotoxic activity.

### Example 4

### Expansion Fold of γδ T Cell Population Cultured by Using Pamidronate

(Culture Using Yssel's Medium)

(1) Expansion of γδ T Cell Population

The expansion of a γδ T cell population was carried out in the same manner as in Example 2-(1), provided that as a basal medium for culture, a medium prepared originally by the applicant having the same composition as an Yssel's medium (hereinafter referred to as “Yssel's medium”) was used in place of IMDM, and that on the fifth day from the initiation of culture, 1 mL of an Yssel's medium containing 10% (v/v) human AB serum was added to each well, to dilute the cell suspension.

(2) Analysis of Ratio of γδ T Cells

The ratio of γδ T cells was analyzed according to flow cytometry for PBMCs prepared in Example 1-(1) and the cells on the fourteenth day from the initiation of culture prepared in Example 4-(1), in the same manner as in Example 1-(4). As a result, the ratio of the γδ T cells was 8.9% for the PBMCs, and the ratio was from 89.7 to 94.3% for the cells on the fourteenth day from the initiation of culture.

(3) Expansion Fold of γδ T Cells

The number of live cells was counted according to trypan blue staining method for the cells on the fourteenth day from the initiation of culture prepared in Example 4-(1). The expansion fold of γδ T cells was calculated in comparison of the counted number with the number of γδ T cells at the initiation of culture, in the same manner as in Example 1-(5). The results are shown in Table 4.

TABLE 4

| | | Expansion Fold of |
|---|---|---|
| | Immobilized | γδ T Cells (Folds) |
| Stimulant | FN Fragment | Fourteenth Day of Culture |
| Pamidronate | Control (Without | ×65.2 |
| | Immobilization of | |
| | FN Fragment) | |
| | CH-296 | ×101.4 |

As shown in Table 4, the group in which the culture equipment immobilized with CH-296 at an early stage of expansion of γδ T cells was used gave the results of a high expansion fold of the γδ T cells, as compared to that of the control group.

### Example 5 Expansion Fold of γδ T Cell Population Cultured Using Zoledronate

(1) Immobilization of FN Fragment (H296-H296)

The immobilization of an FN fragment was carried out in the same manner as in Example 1-(2), provided that a 6-well cell culture plate was used as a plate to be immobilized, and that an ACD-A solution (pH 5.0) containing H296-H296 (final concentration: 3 μg/mL) was added in a volume of 1.2 mL/well each.

(2) Expansion of Cultured γδ T Cell Population

PBMCs prepared in Example 1-(1) were suspended in an RPMI 1640 medium containing 0.25% (w/v) HSA so as to have a concentration of 1×10⁶ cells/mL, to prepare a cell suspension. Thereafter, the above cells were added to a plate to which nothing was immobilized, or the plate immobilized with H296-H296 prepared in Example 5-(1), in a volume of 5 mL/well each. Zoledronic acid hydrate (zoledronate, name of formulation: zometa injection, manufactured NOVARTIS) was added to each well so as to have a final concentration of 1 μM. These plates were incubated at 37° C. in 5% CO₂ (zeroth day of culture).

After 48 hours from the initiation of culture, the cells were removed from the plate, and collected in a 15 mL centrifuge tube (manufactured by Corning), and thereafter the cell suspension was centrifuged at 250×g for 4 minutes. Supernatant of the centrifuged solution (containing zoledronate) was removed, and the cell suspension was then adjusted to a concentration of 1×10⁶ cells/mL with 10HRPMI+L-Gln. The cell suspension was added to a 12-well cell culture plate to which nothing was immobilized. IL-2 was added to each well so as to have a final concentration of 100 U/mL.

On the fourth day of culture, the cell suspension was diluted with a medium for culture so as to have a concentration of 1×10⁶ cells/mL, and IL-2 was added to each well so as to have a final concentration of 100 U/mL.

On the seventh day and the tenth day of culture, the cell suspension was diluted with the medium for culture so as to have a concentration of 0.5×10⁶ cells/mL, and the dilution was transferred to a 6-well cell culture plate to which nothing was immobilized. IL-2 was added to each well so as to have a final concentration of 100 U/mL. The culture was continued up to the fourteenth day from the initiation of culture.

(3) Analysis of Ratio of γδ T Cells

The ratio of γδ T cell was analyzed according to flow cytometry for PBMCs prepared in Example 1-(1) and the cells on the fourteenth day from the initiation of culture prepared in Example 5-(2), in the same manner as in Example 1-(4). As a result, the ratio of γδ T cells was 6.2% for PBMCs, and the ratio was from 89.3 to 91.3% for the cells on the fourteenth day from the initiation of culture.

(4) Expansion Fold of γδ T Cells

The number of live cells was counted according to trypan blue staining method for the cells on the fourteenth day from the initiation of culture prepared in Example 5-(2). The expansion fold of γδ T cells was calculated in comparison of the counted number with the number of γδ T cells at the initiation of culture, in the same manner as in Example 1-(5). The results are shown in Table 5.

TABLE 5

| | | Expansion Fold of |
|---|---|---|
| | Immobilized | γδ T Cells (Folds) |
| Stimulant | FN Fragment | Fourteenth Day of Culture |
| Zoledronate | Control (Without | ×80.8 |
| | Immobilization of | |
| | FN Fragment) | |
| | H296-H296 | ×107.5 |

As shown in Table 5, the group in which the culture equipment immobilized with H296-H296 at an early stage of expansion of γδ T cells was used gave the result of a high expansion fold of γδ T cells during the culture, as compared to that of the control group.

### Example 6

When a bisphosphonic acid compound such as alendronate, risedronate, neridronate, ibandronate, incadronate, olpadronate, solvadronate, minodronate, EB1053, etidronate, clodronate, tiludronate, or medronate is used in place of pamidronate in Example 1, 2, 3, or 4, similar results are obtained.

### Example 7

When a pyrophosphate monoester compound such as 2-methyl-3-butenyl-1-pyrophosphate or 4-hydroxy-3-methyl-2-butenyl-1-pyrophosphate is used in place of IPP in Example 1, similar results are obtained.

### Preparation Example

Preparation Example of a pharmaceutical agent of the present invention is shown below.

The γδ T cell population is prepared in a large scale, on the bases of the methods described in Examples 1 to 7, and the prepared γδ T cell population having a concentration of from 1×10⁴ to 1×10¹¹ cells is suspended in 0.5 to 500 mL of saline, to use the suspension as an injection or infusions.

Alternatively, the γδ T cell population after the preparation is subjected to frozen storage in liquid nitrogen or at −80° C. in a state that the cell population is suspended in a frozen storage solution composed of 49.5% RPMI 1640, 34.0% CP-1, and a 16.5% buminate solution (buminate, 25% human serum albumin solution). After the frozen stored γδ T cell population is rapidly melted in a warm water bath at 37° C., the γδ T cell population is directly used, or suspended in 10 to 500 mL of saline, to use the suspension as an injection or infusions.

This pharmaceutical agent exhibits a cytotoxic activity shown in Example 3, and is effective for the treatment of various diseases mentioned above. Also, this pharmaceutical agent is effective in the treatment of various diseases mentioned above according to adoptive immunotherapy.

### INDUSTRIAL APPLICABILITY

According to the present invention, a method for producing a γδ T cell population is provided. The γδ T cells obtained by the method are preferably used in, for example, immunotherapy. Accordingly, the method of the present invention is expected to greatly contribute to medical fields.

### BRIEF DESCRIPTION OF THE DRAWINGS

FIG. 1 A schematic view showing a domain structure of fibronectin.

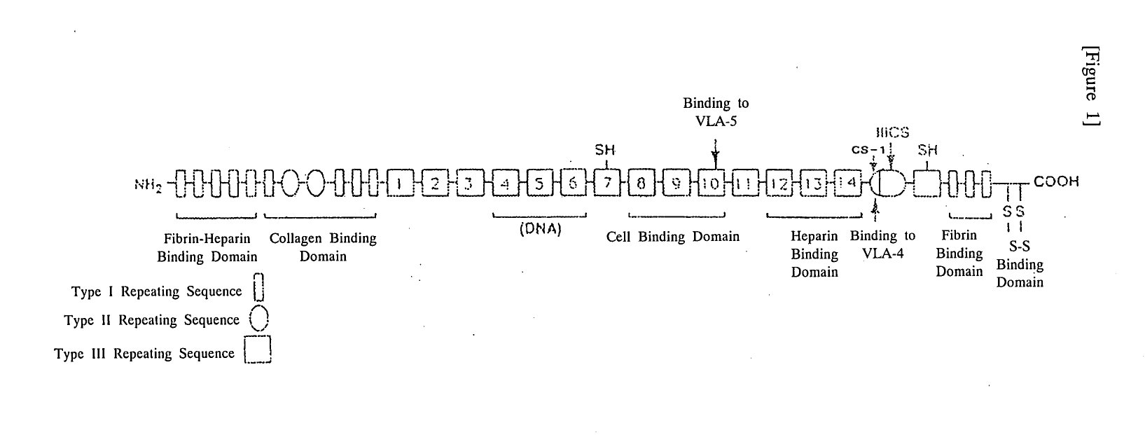

FIG. 2 A view showing a method for preparing H296-H296.

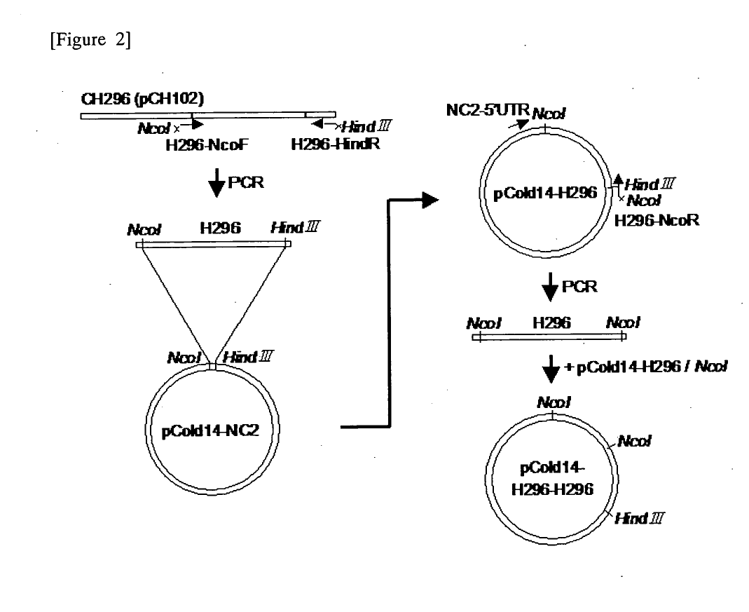

### SEQUENCE FREE TEXT

SEQ ID NO: 1: Partial region of fibronectin named III-8.

SEQ ID NO: 2: Partial region of fibronectin named III-9.

SEQ ID NO: 3: Partial region of fibronectin named III-10.

SEQ ID NO: 4: Partial region of fibronectin named III-11.

SEQ ID NO: 5: Partial region of fibronectin named III-12.

SEQ ID NO: 6: Partial region of fibronectin named III-13.

SEQ ID NO: 7: Partial region of fibronectin named III-14.

SEQ ID NO: 8: Partial region of fibronectin named CS-1.

SEQ ID NO: 9: Fibronectin fragment named C-274.

SEQ ID NO: 10: Fibronectin fragment named H-271.

SEQ ID NO: 11: Fibronectin fragment named H-296.

SEQ ID NO: 12: Fibronectin fragment named CH-271.

SEQ ID NO: 13: Fibronectin fragment named CH-296.

SEQ ID NO: 14: Fibronectin fragment named C-CS1.

SEQ ID NO: 15: Fibronectin fragment named CH-296Na.

SEQ ID NO: 16: Fibronectin fragment named CHV-89.

SEQ ID NO: 17: Fibronectin fragment named CHV-90.

SEQ ID NO: 18: Fibronectin fragment named CHV-92.

SEQ ID NO: 19: Fibronectin fragment named CHV-179.

SEQ ID NO: 20: Fibronectin fragment named CHV-181.

SEQ ID NO: 21: Fibronectin fragment named H-275-Cys.

SEQ ID NO: 22: Fibronectin fragment named H296-H296.

SEQ ID NO: 23: Primer H296-NcoF.

SEQ ID NO: 24: Primer H296-HindR.

SEQ ID NO: 25: Primer H296-NcoR.

SEQ ID NO: 26: Primer NC2-5′ UTR.

1. A method for preparing a γδ T cell population, characterized in that the method comprises the step of culturing a cell population comprising γδ T cells, in the presence of (a) fibronectin, a fibronectin fragment or a mixture thereof and (b) an activating factor of γδ T cells.

2. The method according to claim 1, wherein the step of culturing the cell population in the presence of fibronectin, a fibronectin fragment or a mixture thereof is carried out in the presence of IL-2.

3. The method according to claim 1 or 2, wherein the fibronectin fragment is a polypeptide (m) comprising at least any one of the amino acid sequences shown in SEQ ID NOs: 1 to 8 of Sequence Listing, or a polypeptide (n) comprising at least one amino acid sequence having substitution, deletion, insertion or addition of one or the plural number of amino acids in any one of said amino acid sequences, wherein the polypeptide (n) has a function equivalent to that of said polypeptide (m).

4. The method according to claim 3, wherein the fibronectin fragment is a polypeptide comprising all of the amino acid sequences shown in SEQ ID NOs: 5 to 8 of Sequence Listing.

5. The method according to claim 3, wherein the fibronectin fragment is a polypeptide comprising any one of the amino acid sequences shown in SEQ ID NOs: 9 to 22 of Sequence Listing.

6. The method according to claim 1, wherein the activating factor of γδ T cells is a bisphosphonic acid compound and/or a pyrophosphate monoester compound.

7. The method according to claim 6, wherein the bisphosphonic acid compound is at least one compound selected from the group consisting of pamidronate, alendronate, zoledronate, risedronate, neridronate, ibandronate, incadronate, olpadronate, solvadronate, minodronate, EB1053, etidronate, clodronate, tiludronate and medronate.

8. The method according to claim 6, wherein the pyrophosphate monoester compound is at least one compound selected from the group consisting of isopentenyl pyrophosphate, 2-methyl-3-butenyl-1-pyrophosphate and 4-hydroxy-3-methyl-2-butenyl-1-pyrophosphate.

9. The method according to claim 1, further comprising the step of transducing a foreign gene into the cell population.

10. The method according to claim 9, wherein the foreign gene is transduced using retrovirus vector, adenovirus vector, adeno-associated virus vector, lentivirus vector or Simian virus vector.

11. A γδ T cell population obtained by the method as defined in claim 1.

12. A pharmaceutical agent comprising as an effective ingredient the γδ T cell population obtained by the method as defined in claim 1.

13. A method for treating or preventing a disease, comprising the step of administering to a subject an effective amount of the γδ T cell population obtained by the method as defined in claim 1.

14. Use of the γδ T cell population obtained by the method as defined in claim 1 for the manufacture of a pharmaceutical agent.

15. A γδ T cell population obtained by the method as defined in claim 1 for use in an adoptive immunotherapy.


SEQUENCE LISTING (27 sequences)

SEQ ID NO: 1 - protein, 87 aa, Artificial Sequence
  Partial region of fibronectin named III-8
PTDLRFTNIGPDTMRVTWAPPPSIDLTNFLVRYSPVKNEEDVAELSISPSDNAVVLTNLLPGTEYVVSVSSVYEQHESTPLRGRQKT

SEQ ID NO: 2 - protein, 90 aa, Artificial Sequence
  Partial region of fibronectin named III-9
GLDSPTGIDFSDITANSFTVHWIAPRATITGYRIRHHPEHFSGRPREDRVPHSRNSITLTNLTPGTEYVVSIVALNGREESPLLIGQQST

SEQ ID NO: 3 - protein, 94 aa, Artificial Sequence
  Partial region of fibronectin named III-10
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 4 - protein, 84 aa, Artificial Sequence
  Partial region of fibronectin named III-11
QMQVTDVQDNSISVKWLPSSSPVTGYRVTTTPKNGPGPTKTKTAGPDQTEMTIEGLQPTVEYVVSVYAQNPSGESQPLVQTAVT

SEQ ID NO: 5 - protein, 92 aa, Artificial Sequence
  Partial region of fibronectin named III-12
AIPAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 6 - protein, 89 aa, Artificial Sequence
  Partial region of fibronectin named III-13
NVSPPRRARVTDATETTITISWRTKTETITGFQVDAVPANGQTPIQRTIKPDVRSYTITGLQPGTDYKIYLYTLNDNARSSPVVIDAST

SEQ ID NO: 7 - protein, 90 aa, Artificial Sequence
  Partial region of fibronectin named III-14
AIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 8 - protein, 25 aa, Artificial Sequence
  Partial region of fibronectin named CS-1
DELPQLVTLPHPNLHGPEILDVPST

SEQ ID NO: 9 - protein, 274 aa, Artificial Sequence
  Fibronectin fragment named C-274
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEID

SEQ ID NO: 10 - protein, 271 aa, Artificial Sequence
  Fibronectin fragment named H-271
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 11 - protein, 296 aa, Artificial Sequence
  Fibronectin fragment named H-296
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 12 - protein, 549 aa, Artificial Sequence
  Fibronectin fragment named CH-271
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 13 - protein, 574 aa, Artificial Sequence
  Fibronectin fragment named CH-296
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 14 - protein, 302 aa, Artificial Sequence
  Fibronectin fragment named C-CS1
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSDELPQLVTLPHPNLHGPEILDVPST

SEQ ID NO: 15 - protein, 658 aa, Artificial Sequence
  Fibronectin fragment named CH296Na
MPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 16 - protein, 367 aa, Artificial Sequence
  Fibronectin fragment named CHV-89
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMNVSPPRRARVTDATETTITISWRTKTETITGFQVDAVPANGQTPIQRTIKPDVRSYTITGLQPGTDYKIYLYTLNDNARSSPVVIDAST

SEQ ID NO: 17 - protein, 368 aa, Artificial Sequence
  Fibronectin fragment named CHV-90
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 18 - protein, 370 aa, Artificial Sequence
  Fibronectin fragment named CHV-92
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 19 - protein, 457 aa, Artificial Sequence
  Fibronectin fragment named CHV-179
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMNVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 20 - protein, 459 aa, Artificial Sequence
  Fibronectin fragment named CHV-181
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDAST

SEQ ID NO: 21 - protein, 276 aa, Artificial Sequence
  Fibronectin fragment named H-275-Cys
MAASAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTC

SEQ ID NO: 22 - protein, 594 aa, Artificial Sequence
  Fibronectin fragment named H296-H296
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTAMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 23 - DNA, 32 bp, Artificial Sequence
  Synthetic primer H296-NcoF
gagcggataaccatggctattcctgcaccaac

SEQ ID NO: 24 - DNA, 36 bp, Artificial Sequence
  Synthetic primer H296-HindR
gagcggataaaagcttttatgtggaaggaacatcca

SEQ ID NO: 25 - DNA, 34 bp, Artificial Sequence
  Synthetic primer H296-NcoR
gattacctaccatggctgtggaaggaacatccaa

SEQ ID NO: 26 - DNA, 20 bp, Artificial Sequence
  Synthetic primer NC2-5′ UTR
gcatatccagtgtagtaagg

SEQ ID NO: 27 - protein, 4 aa, Artificial Sequence
  Synthetic peptide
RGDS